(19) United States
(12) Patent Application Publication
(10) Pub. No.: US 2024/0382400 A1
(43) Pub. Date: Nov. 21, 2024

(54) SKIN CARE COMPOSITION COMPRISING HYDROXYCINNAMIC ACID AND NIACINAMIDE DERIVATIVE

(71) Applicant: The Procter & Gamble Company, Cincinnati, OH (US)

(72) Inventors: Matthew Clair Ehrman, Singapore (SG); Wan ting Chung, Singapore (SG); Shikhar Gupta, Singapore (SG); John Erich Oblong, Loveland, OH (US)

(21) Appl. No.: 18/661,776

(22) Filed: May 13, 2024

Related U.S. Application Data
(60) Provisional application No. 63/466,352, filed on May 15, 2023.

(51) Int. Cl. A61K 8/365; A61K 8/67; A61Q 19/00

(52) U.S. Cl. CPC A61K 8/365; A61K 8/675; A61Q 19/00

(57) ABSTRACT

A low-pH, aqueous skin care composition that includes from about 0.1% to about 10% of a hydroxycinnamic acid (HCA), from about 0.1% to about 10% of a niacinamide derivative having a free binding energy with HCA of −10 Kcal/mol or more, and water. The composition can have a pH of less than 5.0. The composition provides reduced crystal formation of HCA.

### FIELD

The present disclosure relates to a low-pH, aqueous skin care composition, comprising: a) from about 0.1% to about 10% of a hydroxycinnamic acid (HCA); and b) from about 0.1% to about 10% of a niacinamide derivative having a free binding energy with HCA of −10 Kcal/mol or more; and c) water, wherein the pH of the composition is less than 5.0. The composition provides reduced crystal formation of HCA.

### BACKGROUND

Skin is the first line of defense against environmental insults that would otherwise damage sensitive underlying tissue and organs. Additionally, skin plays a key role in a person's physical appearance. Not surprisingly, most people would like to have heathy, younger looking skin. Unfortunately for some people, the tell-tale signs of aging such as thinning skin, wrinkles, and age spots are an undesirable reminder of the disappearance of youth. The desire for healthy, younger looking skin has led to the development of numerous skin care products marketed to treat the various skin conditions associated with aging and unhealthy skin. These skin care products typically include one or more active ingredients for treating a skin condition of interest.

Antioxidants are commonly used in skin care products as active ingredients, for example, as disclosed in WO 2008/73684. And hydroxycinnamic acids (HCAs) are particularly well-known antioxidants, for example, as described in WO 2018/081790. However, the use of hydroxycinnamic acids in skin care composition can be problematic. For example, HCAs are relatively insoluble in water, which can lead to the undesirable formation of HCA crystals in the product. Further, when HCA is used at neutral pH (e.g., pH 5.0-8.0), which is common for skin care compositions, the HCA may oxidize or degrade, causing undesirable color changes, odors and/or reduced efficacy of the product. In some instances, formulating the composition at a lower pH may help stabilize the HCA, but it also lowers HCA solubility.

There remains a need to provide low-pH compositions containing HCA, with reduced crystal formation of HCA.

### SUMMARY

Disclosed herein is a low-pH, aqueous skin care composition, comprising:
a) from about 0.1% to about 10% of a hydroxycinnamic acid (HCA); and
b) from about 0.1% to about 10% of a niacinamide derivative having a free binding energy with HCA of −10 Kcal/mol or more; and
c) water, wherein the pH of the composition is less than 5.0.

The composition provides reduced crystal formation of HCA by the use of niacinamide derivatives having a specific free binding energy with HCA.

Hydroxycinnamic acids such as para-coumaric acid (hereinafter also called as p-coumaric acid), tend to exhibit undesirable solubility and/or stability characteristics in conventional skin care products. The insolubility of HCA is even worse in low-pH compositions. However, it was recently discovered that low-pH compositions can improve the efficacy of certain skin care actives such as niacinamide (see, U.S. Pat. No. 10,874,600). However, it has been surprisingly found that niacinamide and HCA especially p-coumaric acid predominately form co-crystals in low pH compositions such as in compositions at pH less than 5, especially at pH of from about 3.5 to less than 5, especially when niacinamide is included in the composition at a higher level such as 1% or more. It has been further surprisingly found that the free binding energy of niacinamide and HCA, especially p-coumaric acid, may be lower than HCA alone, which could be reflecting the observation of predominately co-crystal formation between niacinamide and HCA, especially p-coumaric acid. Hereinafter, co-crystals between niacinamide and HCA, especially p-coumaric acid, will be called HCA crystals for simplicity.

Then, it has been surprisingly found by the present inventors that HCA, especially p-coumaric acid, and specific niacinamide derivatives having free binding energy of −10 kcal/mol or more, form reduced co-crystals or does not form co-crystals, at pH less than 5, even at pH of from about 3.5 to less than 5, even when such niacinamide derivatives are included in the composition at a higher level such as 1% or more. Additionally, it has been found that, among such specific niacinamide derivatives, methyl niacinamide and salts thereof provides improved skin care benefit, especially equivalent and/or improved efficacy to niacinamide shown by the PGE2 anti-inflammation assay.

### DETAILED DESCRIPTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The compositions can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Apply” or “application,” as used in reference to a composition, means to apply or spread the compositions onto a human skin surface such as the epidermis.

“Free Binding Energy” is defined as the Gibbs free energy difference between bound and unbound states of two molecules.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications.

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein. In a specific example, an effective amount of a vitamin B₃ compound is an amount sufficient to improve the health and/or appearance of psoriatic skin during a treatment period. In some instances, an effective amount may be demonstrated using ex vivo and/or in vitro methods.

“Hydroxycinnamic acid” (HCA) refers to a class of aromatic acids or phenylpropanoids having a C6-C3 skeleton that are hydroxy derivatives of cinnamic acid. Some non-limiting examples of HCA are caffeic acid, cichoric acid, cinnamic acid, chlorogenic acid, diferulic acids, coumaric acids (p-, o-, and m-), ferulic acid, sinapinic acid, sinapic acid, and α-cyano-4-hydroxycinnamic acid.

“Improve the appearance of” means providing a measurable, desirable change or benefit in skin appearance, which may be quantified, for example, by a decrease in redness, inflammation, and/or plaque scales.

“Low-pH” means a pH of less than 5.0 (e.g., 1.5 to 4.9, 2.0 to 4.5, 2.5 to 4.0, or 3.5 to 4.0). A suitable method of determining the pH of a composition is described in more detail below.

“Neutral pH” means a pH of 5.0 to 8.0.

“Safe and effective amount” means an effective amount of an ingredient that is low enough to avoid serious side effects (within the scope of sound medical judgment).

“Skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Treatment period,” as used herein, means the length of time and/or frequency that a material or composition is applied to a target skin surface.

### Composition

The skin care compositions described herein are low-pH compositions intended for topical application to human skin to improve the appearance, health, and/or function of skin. The present compositions may be used for non-therapeutic (i.e., cosmetic) treatment of a variety of skin conditions. For example, the low-pH composition may be particularly suitable for improving the appearance of skin, especially fine lines, wrinkles, hyperpigmented spots, uneven skin tone, sallow looking skin and/or skin stability. Improved skin stability means to provide reduced day-to-day fluctuation of skin condition.

The skin care compositions described herein is a low-pH, aqueous skin care composition, comprising:
a) from about 0.1% to about 10% of a hydroxycinnamic acid (HCA); and
b) from about 0.1% to about 10% of a niacinamide derivative having a free binding energy with HCA of −10 Kcal/mol or more; and
c) water, wherein the pH of the composition is less than 5.0.

The composition provides reduced crystal formation of HCA by the use of niacinamide derivatives having a specific free binding energy with HCA. Preferably, the composition herein is free of HCA crystals. A suitable method for determining whether a composition is free of HCA crystals and/or characterizing HCA crystals in a composition is described in more detail below.

In some aspects, the composition may include a silicone emulsifier, a polymer thickener that can tolerate low-pH environments, a low molecular weight silicone fluid, an acid-salt pH-buffering system (e.g., a lactic acid/sodium lactate buffering system), and/or other ingredients commonly found in topical skin care compositions. It is believed, without being limited by theory, that the combinations of ingredients disclosed herein provides a stable and efficacious skin care composition that has good feel properties and is gentle on skin.

The low-pH skin care compositions herein can be made by mixing the ingredients with a dermatologically acceptable carrier using conventional methods known to those skilled in the art. The compositions may be provided in various product forms such as solutions, suspensions, lotions, creams, gels, toners, sticks, sprays, aerosols, ointments, cleansing liquid washes and solid bars, pastes, foams, mousses, shaving creams, wipes, strips, patches, electric-powered patches, hydrogels, film-forming products, facial and skin masks (with and without insoluble sheet), and the like. The composition form may follow from the particular dermatologically acceptable carrier chosen.

In some instances, the low-pH skin care composition herein may be in the form of an essence. An essence is a form of topical skin care composition in a relatively concentrated formula that typically has a lower viscosity than a conventional cream- or lotion-type skin care composition. An essence may be provided in the form of a low viscosity fluid that is marketed to specifically target a particular skin condition and/or be used in the first step of a skin care regimen. The skin care essence products herein can have a dynamic viscosity of 1 centipoise (cP) to 15,000 cP at 25° C. (e.g., 50 cP to 10,000 cP or 100 cP to 7,500 cP, 200 cp to 5,000 cp, or 300 cp to 2,500 cp). A method of determining the viscosity of the low-pH compositions is described in more detail in the Methods section below.

### Hydroxycinnamic Acid (HCA)

The low-pH skin care compositions herein include a safe and effective amount of HCA. The HCA may be present in the composition at 0.1% to 10% (e.g., 0.5% to 5% or 1% to 4%). Hydroxycinnamic acids are generally recognized as antioxidant phenolic compounds, which can be found in plants, mainly as a component of cell walls. See, H. K. Kuzaki et al., J. Agric. Food Chem., 50, 2161-68 (2002).

In some aspects, it may be desirable to select coumaric acid for use in the low-pH composition, especially p-coumaric acid (a.k.a. 4-HCA). P-coumaric acid has the following structure:

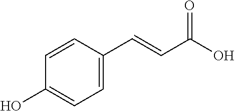 [SMILES: O=C(O)/C=C/c1ccc(O)cc1]

In other aspects, it may be desirable to use a mixture of two or more HCAs such as, for example, a mixture of coumaric acid and ferulic acid. In these aspects, the coumaric acid and ferulic acid may be present at a weight ratio of 2:1 to 1:2 (e.g., 1:1). A particularly suitable example of an HCA material suitable for use herein is LIPOBRITE available from Vantage Personal Care.

### Niacinamide Derivatives Having a Higher Free Binding Energy

The present compositions include niacinamide derivatives having a free binding energy with HCA of −10 Kcal/mol or more, preferably −7.5 kcal/mol or more, and more preferably −5 kcal/mol or more, in view of reduced crystallization.

Free binding energy is measured as follows:

### Free Binding Energy Calculation

All calculations were performed using Gaussian 09 program package. The optimization of gas-phase structure of each molecular system (individual molecules & complexes) was obtained using the density functional theory (DFT) method involving the Becke 3-parameter exchange functional together with the Lee-Yang-Parr correlation functional (B3LYP). Vibrational frequencies were calculated to ensure that the optimized geometries were local minima. The energies, electronic & thermodynamic parameters and charges on the optimized p-coumaric acid (HCA) and different HCA—Niacinamide/Derivative complex structures were calculated at the 6-311++G(d, p) levels of theory. The Free binding energy between HCA and Niacinamide/derivatives was calculated as the difference between the energy of the complex and that of the sum of the individual monomers. The Free binding energy was calculated from the below relation:

$$Δ {E}_{AB} = {E}_{AB} - ( {E}_{A} + {E}_{B} )$$

Where E_{AB}, E_{A}, and E_{B} are the energies of the complex, the individual monomers respectively. In addition, the basis set superposition error (BSSE) correction was calculated to obtain accurate Free binding energies.

For example, the below table shows free binding energy of p-coumaric acid with niacinamide and some derivatives.

| | Free binding energy with |
|---|---|
| | p-coumaric acid, (ΔG) |
| | Kcal/mol |
| Niacinamide | −14.44 |
| Methyl niacinamide* | −4.53 |
| Nicotinamide Mononucleotide | −3.87 |
| Nicotinamide Adenine Dinucleotide | −3.37 |

*Also, for salts of Methyl niacinamide, as such salts exists as methyl niacinamide in the compositions and in the condition measuring the free binding energy.

Preferably, the niacinamide derivatives useful herein have a pyridine portion, in which a nitrogen is protonated in the composition. Preferably, niacinamide derivatives useful herein are selected from the group consisting of Methyl niacinamide, salts of methyl niacinamide, Nicotinamide Mononucleotide, Nicotinamide Adenine Dinucleotide, and mixtures thereof. More preferably niacinamide derivative is selected from the group consisting of Methyl niacinamide, salts of methyl niacinamide, and mixtures thereof, in view of skin care benefit, especially its equivalent and/or improved efficacy to niacinamide in PGE2 anti-inflammation in-vitro. Salts of methyl niacinamide can be any salts given the counter-ion in the aqueous phase dissociates and doesn't affect the free binding energy, including, for example methyl niacinamide chloride. As stated above, salts of methyl niacinamide exists as methyl niacinamide in the compositions and in the condition measuring the free binding energy.

The present compositions include 0.1% to 10% (e.g., 0.5% to 5% or 1% to 4%) of a niacinamide derivatives for regulating a variety of skin condition, for example, as described in U.S. Pat. No. 5,939,082.

In some instances, it may be desirable for the ring nitrogen of the vitamin B₃ compound to be uncomplexed (e.g., chemically unbound and/or unhindered) in the composition and/or prior to application to a target skin surface. For example, the compositions herein may be free or substantially free (i.e., less than 3%, 2%, 1% or even less than 0.5%) of a salt or complex of a vitamin B₃ compound. Exemplary approaches to minimizing or preventing the formation of undesirable salts and/or complexes include omission of materials that form substantially irreversible or other undesirable complexes with the vitamin B₃ compound in the composition, pH adjustment, ionic strength adjustment, the use of surfactants, and practicing formulation processes wherein the vitamin B₃ compound and materials which complex therewith are in different phases.

### Niacinamide and its Derivatives Having Lower Free Binding Energy with HCA

The composition may include Niacinamide and its derivatives having lower free binding energy with HCA, in addition to the above niacinamide derivatives having higher free binding energy with HCA.

If included, the weight ratio between “niacinamide and its derivatives having lower free binding energy with HCA” and “niacinamide derivatives having higher free binding energy with HCA” is, preferably up to about 7:3, more preferably up to about 6:4, still more preferably up to about 5:5, further more preferably up to about 4:6, even more preferably up to about 3:7.

If included, the blend of “niacinamide and its derivatives having lower free binding energy with HCA” and “niacinamide derivatives having higher free binding energy with HCA” provide, preferably more than 110% Relative Saturated Solubility of HCA alone, more preferably more than 120%, still more preferably more than 130%, even more preferably more than 140% Relative Saturated Solubility of HCA alone.

Below tables I-III are for explaining saturated solubility and relative saturated solubility:

TABLE I

| | A | B | C | D |
|---|---|---|---|---|
| Water Purified | 79.9 | 79.9 | 79.9 | 79.9 |
| Niacinamide | — | 2 | — | — |
| Methyl Niacinamide Chloride | — | — | 2 | — |
| Nicotinamide Mononucleotide | — | — | — | 2 |
| P-Coumaric Acid | 2 | 2 | 2 | 2 |
| Ferulic Acid | — | — | — | — |
| pH * | 4.5 | 4.5 | 4.5 | 4.5 |
| Saturated Solubility (mg/mL) | 1.075 | 0.897 | 2.220 | 1.482 |
| of HCA | | | | |
| Relative Saturated Solubility | Control | 83.4% | 206.5% | 137.9% |
| versus HCA control (A) | | (versus | (versus | (versus |
| | | A) | A) | A) |

* pH balanced with HCL/NaOH solutions to the target pH listed.

TABLE II

| | E | F | G |
|---|---|---|---|
| Water Purified | 79.9 | 79.9 | 79.9 |
| Niacinamide | — | 2 | — |
| Methyl Niacinamide Chloride | — | — | 2 |
| Nicotinamide Mononucleotide | — | — | — |
| P-Coumaric Acid | 2 | 2 | 2 |
| Ferulic Acid | — | — | — |
| pH * | 3.5 | 3.5 | 3.5 |
| Saturated Solubility (mg/mL) of | 0.681 | 0.719 | 1.15 |
| HCA | | | |
| Relative Saturated Solubility versus | Control | 105.6% | 168.9% |
| HCA control (E) | | (versus E) | (Versus E) |

TABLE III

| | H | I | J | K |
|---|---|---|---|---|
| Water Purified | 79.9 | 79.9 | 79.9 | 79.9 |
| Niacinamide | — | 2 | — | — |
| Methyl Niacinamide | — | — | 2 | — |
| Chloride | | | | |
| Nicotinamide | — | — | — | 2 |
| Mononucleotide | | | | |
| P-Coumaric Acid | — | — | — | — |
| Ferulic Acid | 2 | 2 | 2 | 2 |
| pH * | 4.5 | 4.5 | 4.5 | 4.5 |
| Saturated Solubility | 1.492 | 1.822 | 3.593 | 1.936 |
| (mg/mL) of HCA | | | | |
| Relative Saturated | Control | 122.2% | 240.8% | 129.8% |
| Solubility versus | | (Versus H) | (Versus H) | (Versus H) |
| HCA control (H) | | | | |

Preferably, the composition is substantially free of Niacinamide and its derivatives having lower free binding energy with HCA, i.e., contains 0.1% or less, more preferably 0.05% or less of such materials. Still more preferably, the composition is free of such materials, i.e., contains 0% of such materials.

### Low-pH Acid Buffering System

When providing a low-pH composition for topical application to skin, it is highly preferred to include a buffering system to help maintain the pH of the composition for a period of time after it is applied to the skin (e.g., up to 5 minutes or more). On average, human skin pH typically ranges from about 5.0 to 6.0. To maintain this pH, human skin has evolved a natural buffering system that resists changes to pH. Thus, when a low-pH composition is applied to the skin, the skin's natural buffering system will try to adjust the pH of the composition to match the natural pH of the skin. Without the addition of the buffering agent, the low-pH composition may not be able to provide the desired skin care benefit. Accordingly, the compositions herein may include a low-pH acid buffering system.

The buffering agent may be selected according to the acid(s) that is used to lower the pH of the low-pH compositions herein. For example, lactic acid and gluconic acid may be used to lower the pH of the composition because they are generally considered to be gentler on skin (i.e., lower risk of irritation) compared to other alpha hydroxy acids. In this example, sodium lactate or sodium gluconate would then be selected to provide the acid/salt pH buffer system. The buffering agent may be present in the low-pH composition at 0.25% to 4% (e.g., 0.5% to 3%, 0.75% to 2% or 1% to 1.75%). A non-limiting example of a suitable low-pH buffer system for use herein is disclosed in co-pending U.S. Ser. No. 16/891,491. Of course, it is to be appreciated that the present composition may optionally include other pH buffers known for use in skin care compositions.

### Thickeners

The composition preferably includes a polymer thickener that can tolerate a low-pH, electrolytic environment. That is, the thickener will not lose its ability to thicken or stabilize the composition at low-pH in the presence of an acid-salt buffering system. Some conventional neutralized thickeners are known to degrade and/or lose the ability to suitably thicken a composition at lower pH and/or in the presence of an acid-salt buffer (e.g., sodium lactate). For example, some neutralized thickeners degrade in a low-pH environment. On the other hand, fatty alcohol thickeners such as cetyl alcohols and stearyl alcohols are generally stable at low pH but tend to impart an undesirable cloudiness or opacity to the composition when it is in the form of an essence, serum, or the like. It has also been found that certain anionic polymeric thickeners can provide suitable tolerance to low-pH environments but cannot tolerate buffer systems due to combination of acid and salt. Thus, in some instances, the low-pH composition described herein may be free or substantially free of neutralized thickeners, fatty alcohol thickeners, and anionic thickeners. The thickener may be present at 0.0001% to 25% (e.g., 0.001% to 20%, 0.01% to 10%, 0.5% to 7%, or 1% or 5%) by weight of the composition.

Other nonlimiting examples of thickeners or water structuring agents that may be used alone or in combination herein include natural or synthetic gums, polysaccharides, carboxylic acid polymers, polyacrylamide polymers, sulfonated polymers, and copolymers of these. Further examples include modified gums, celluloses, and superabsorbent polymers. The term “superabsorbent polymer” is understood to mean a polymer which is capable, in its dry state, of spontaneously absorbing at least 20 times its own weight of aqueous fluid, in particular of water and especially of distilled water. Suitable polysaccharides include alkyl hydroxyalkyl cellulose ethers, such as hydroxypropylmethylcellulose stearoxy ether. This material is sold under the tradename of SANGELOSE 60 L and 90 L from Daido Chemical Corp. Another suitable polysaccharide includes hydrophobically modified starch, such as Potato modified starch. This material is sold under the tradename of STRUCTURE SOLANACE by Nouryon. Another polymer includes crosslinked polymers, the monomers of which are at least partially composed of acryloyldimethyltaurate monomers, such as, for example sodium polyacryloyldimethyl taurate, sold under the tradename of ARISTOFLEX SILK, from Clariant.

It has now been found that certain anionic polymeric thickeners can provide suitable tolerance to low-pH environments and the desired feel and opacity properties to the composition. Thus, a particularly suitable example of an anionic thickener is polyacrylate crosspolymer-6, which is commercially available as SEPIMAX ZEN from Seppic, France.

### Low Molecular Weight Silicone Fluid

In some instances, an anionic polymeric thickener may impart an undesirable tacky feel when the low-pH composition is applied to a target portion of skin. It has been found that the addition of a low molecular weight silicone fluid can reduce or prevent this tacky feel. The molecular weight of a silicone fluid depends on the length of its silicone polymer chain(s), which is also directly proportional to the viscosity of the silicone fluid. Thus, the low molecular weight silicone fluids suitable for use in the present low-pH composition have a kinematic viscosity of 100 cSt or less at 25° C. (e.g., 1 cSt to 90 cSt, 5 cSt to 50 cSt, or even 10 cSt to 30 cSt). Kinematic viscosity is a common method of classifying silicone fluids and can be obtained from the supplier of the material. A particularly suitable example of a low molecular weight silicone fluid is 5 cSt dimethicone fluid. As used herein, “dimethicone” means a polydimethylsiloxane compound having the formula:

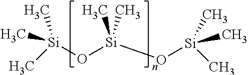

### Dermatologically Acceptable Carrier

The low-pH compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous, or anhydrous), dispersions, emulsions, and solid forms (e.g., gels, sticks, flowable solids, or amorphous materials). In some instances, the dermatologically acceptable carrier is in the form of an emulsion. The emulsion may have a continuous aqueous phase (e.g., an oil-in-water or water-in-oil-in-water emulsion) or a continuous oil phase (e.g., water-in-oil or oil-in-water-in-oil emulsion). The oil phase may comprise silicone oils, non-silicone oils such as hydrocarbon oils, esters, ethers, and mixtures thereof. The aqueous phase typically comprises water and water-soluble ingredients (e.g., water-soluble moisturizing agents, conditioning agents, anti-microbials, humectants and/or other skin care actives). However, in some instances, the aqueous phase may comprise components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s).

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable, hydrophilic diluents. As used herein, “diluent” includes materials in which the vitamin B₃ compound can be dispersed, dissolved, or otherwise incorporated. Hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., molecular weight of 425 to 2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Emulsifier

When the low-pH composition herein is in the form of an emulsion (e.g., oil-in-water emulsion), it may be desirable to include an emulsifier to stabilize the emulsion (i.e., prevent the emulsion from phase separating). The emulsifier may be present in the composition at 0.01% to 10% (e.g., 0.05% to 5% or 0.1% to 2%). The emulsifiers may be nonionic, anionic, or cationic. In some instances, the emulsifier may be a silicone emulsifier. Some non-limiting examples of emulsifiers that may be suitable for use herein are disclosed in U.S. Pat. Nos. 3,755,560; 4,421,769; and Mccutcheon's Detergents and Emulsifiers, North American Edition, pages 317-324 (1986).

Some other non-limiting examples of emulsifiers that may be suitable for use herein include ethers of polyglycols and of fatty alcohols, esters of polyglycols and of fatty acids, ethers of polyglycols and of fatty alcohols which are glycosylated, esters of polyglycols and of fatty acids which are glycosylated, ethers of C12-30 alcohols and of glycerol or of polyglycerol, esters of C12-30 fatty acids and of glycerol or of polyglycerol, ethers of oxyalkylene-modified C12-30 alcohols and of glycerol or polyglycerol, ethers of C1-230 fatty alcohols comprising and of sucrose or of glucose, esters of sucrose and of C1230 fatty acids, esters of pentaerythritol and of C12-30 fatty acids, esters of sorbitol and/or of sorbitan and of C12 30 fatty acids, ethers of sorbitol and/or of sorbitan and of alkoxylated sorbitan, ethers of polyglycols and of cholesterol, esters of C12-30 fatty acids and of alkoxylated ethers of sorbitol and/or sorbitan, and combinations thereof. A particularly useful class of emulsifiers is polyethylene glycol ethers of lauryl alcohol such as laureth-1 through laureth-50 (e.g., laureth-4). Still other examples of emulsifiers include ethers of glycerol, polyglycerol, sucrose, glucose, or sorbitol; esters of glycerol, polyglycerol, sucrose, glucose, or sorbitol; and mixtures thereof. Other particularly useful classes of emulsifiers are the alkyl esters of sorbitol and sorbitol anhydrides such as polysorbate 20, polysorbate 21, and polysorbate 40.

In some aspects, it may be desirable to include a linear or branched silicone emulsifier in the low-pH composition. Particularly useful silicone emulsifiers include polyether modified silicones such as KF-6011, KF-6012, KF-6013, KF-6015, KF-6015, KF-6017, KF-6043, KF-6028, and KF-6038 and polyglycerolated linear or branched siloxane emulsifiers such as KF-6100, KF-6104, and KF-6105; all from Shin-Etsu. A particular suitable emulsifier for use herein is PEG-11 methyl ether dimethicone, which is available from Shin-Etsu as KF-6011. Surprisingly, it was discovered that the PEG-11 methyl ether dimethicone emulsifier further reduced the tacky feel of the anionic polymer thickener, thereby improving the overall feel of the low-pH composition. The emulsifier may be present at an amount of 0.1% to 10% (e.g., 1% to 5%, or 2% −4%).

### Co-Solvent

In some aspects, the compositions herein may include a short chain dihydric alcohol (e.g., glycol) co-solvent to help solubilize the HCA. However, when glycol is selected as the co-solvent, it can be important to limit the amount of glycol to less than 25% (e.g., less than 20%, 17%, 15%, or even less than 10%) to reduce the risk of imparting undesirable feel characteristics to the composition having (e.g., sticky feeling or greasy feeling). Some non-limiting examples of glycols that may be suitable for use herein are propylene glycol, dipropylene glycol, butylene glycol, pentylene glycol, hexylene glycol, ethoxydiglycol, and C2-C6 polyethene glycols (e.g., PEG-3, PEG-4, PEG-4 methyl ether), and combinations thereof.

### Antioxidant

The low-pH composition herein may include an antioxidant to combat HCA oxidation and/or degradation. The antioxidant, when included, may be present at 0.001% to 3% (e.g., 0.01% to 2%, 0.05% to 1%, or 0.1% to 0.5%). Some non-limiting examples of antioxidants that may be suitable for use herein are sodium sulfite, sodium bisulfite sodium metabisulfite, and butylated hydroxytoluene.

### Hydrotrope

The compositions herein may include 0.1% to 10% (e.g., 0.5% to 5% or 1% to 3%) of a hydrotrope to enhance the water solubility of HCA. It is preferred that the weight ratio between HCA and the hydrotrope is from about 2:1 to about 1:20, more preferably from about 1:1 to about 1:10, still more preferably from about 1:1 to about 1:5.HCA compounds generally exhibit especially poor solubility in a low-pH aqueous composition, such as the low-pH compositions described herein. For example, p-coumaric acid has a solubility of approximately 345 mg/mL in water at pH 7.0 and 20° C. and a solubility of 4 mg/mL in water at pH 3.0 and 20° C. Without being limited by theory, it is believed that the decreased solubility of an HCA at lower pH is due to reduced ability of the HCA to undergo the acid dissociation that forms the conjugate base species observed at higher pH levels. At pH 7, p-coumaric acid exists at approximately 99.6% in its conjugate base form; whereas at pH 3, the conjugate base form is only present at approximately 13.4%. As a result of its relatively poor solubility, HCA tends to form crystals by itself (hereinafter HCA mono crystals) in an aqueous, low-pH skin care composition, even without the presence of niacinamide. HCA mono crystals can impart an undesirable feel to the composition during use (e.g., a rough or grainy feel) and/or may decrease the efficacy of the HCA and/or other ingredients in the composition. This can create an undesirable consumer perception of poor product quality.

The hydrotrope useful herein are those selected from the group consisting of: phenolic acid, derivatives of phenolic acid, phenolic alcohol, derivatives of phenolic alcohol, and mixtures thereof; wherein the hydrotrope has a pKA less than the formula pH; and wherein the hydrotrope has the partition coefficient (logP) of less than 3.0.

The hydrotrope should have a pKA less than the formula pH and a sufficient aqueous phase solubility at the formula pH to be fully soluble. Having an acidic pKa that is less than the pH of the composition can be especially important for low-pH compositions due to the reduced solubility of HCA at low pH. For example, sodium salicylate, which has a pKa of 2.8, may be a suitable hydrotrope for use in a composition with a pH of 3.8 because the carboxylic acid primarily exists in the conjugate base form. In contrast, cinnamic acid or sodium cinnamate, which have an acidic pKa of 4.32, would not be effective hydrotropes in this example. Furthermore, the hydrotrope should not form crystals between hydrotrope and the target HCA. Additionally, the hydrotrope should be selected to help improve the sensory appeal of the composition by reducing the need for other solubilizing agents, such as glycols. Some non-limiting examples of hydrotropes that may be suitable for use herein are salicylic acid, 2, 4 dihydroxybenzoic acid, 2, 3 dihydroxybenzoic acid, 3-methoxy salicylic acid, salts of these, or a combination thereof. Other non-limiting examples of hydrotropes that may be suitable for use herein are disclosed in PCT Publication WO 2018/081790.

The hydrotropes useful herein have the partition coefficient (logP) of less than 3.0. The partition coefficient was calculated using the ACD Consensus algorithm calculations (http://perceptahelp.acdlabs.com/help_v2020/index.php/LogP#Consensus_LogP). Below are some examples of ingredients and the partition coefficients are:

| CAS | | | |
|---|---|---|---|
| Number | SMILES | Name | logP¹ |
| 58-08-2 | Cn1cnc2c1c(═O)n(C)c(═O)n2C | Caffeine | 0.283 |
| 538-42-1 | [O—]C(═O)/C═C/c1ccccc1•[Na+] | Sodium Cinnamate | 2.317 |
| 99-50-3 | C1═CC(═C(C═C1C(═O)O)O)O | 3,4-Dihydroxybenzoic | 1.021 |
| | | Acid | |
| 54-21-7 | OC(═O)c1ccccc1O•[Na] | Sodium salicylate | 2.371 |
| 89-86-1 | C1═CC(═C(C═C1O)O)C(═O)O | 2,4-Dihydroxybenzoic | 1.744 |
| | | Acid | |
| 877-22-5 | COC1═CC═CC(═C1O)C(═O)O | 3-Methoxysalicylic acid | 1.976 |
| 303-38-8 | C1═CC(═C(C(═C1)O)O)C(═O)O | 2,3-Dihydroxybenzoic | 1.534 |
| | | acid | |
| 70424-62-3 | CCCCCCCC(═O)OC1═CC═CC═C1C(═O)O | capryloyl salicylic acid | 4.599 |
| 7400-08-0 | c1cc(ccc1C═CC(═O)O)O | 4-hydroxycinnamic acid | 1.69 |

¹logP ACD, Consensus algorithm

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition at low-pH, especially pH sensitive ingredients like niacinamide, salicylates and peptides. In some instances, it may be desirable to select skin care actives that function via different biological pathways so that the actives do not interfere with one another, which could reduce the efficacy of both agents. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Method of Use

The low-pH compositions herein are formulated for topical application to skin. The method of using the present low-pH composition involves identifying a target portion of skin on a person in need of treatment or where treatment is desired (e.g., portions of skin exhibiting uneven tone, sallowness, hyperpigmented spots, fine lines or wrinkles, skin stability) and applying an effective amount of the low-pH composition to the target portion of skin over the course of a treatment period. The effective amount of composition may vary based on the skin benefit desired by the user and/or the size of the treatment area. In some instances, the effective amount may range from 0.1 g to 5 g (e.g., 0.2 g to 4 g, 0.3 g to 2 g, or even 0.5 g to 1 g). The target portion of skin may be on a facial skin surface such as the forehead, perioral, chin, periorbital, nose, and/or check) or another part of the body (e.g., hands, arms, legs, back, chest). In some instances, a target portion of skin may be selected that does not currently exhibit signs of skin aging but is an area of skin that commonly exhibits such features with age. In these instances, the low-pH composition may be used to help prevent the occurrence of such undesirable skin features.

The composition may be applied locally to the target portion of skin in need of treatment and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. When used according to the methods herein, the present compositions may improve the appearance and/or function of skin, for example, by improving skin texture. Improvements in skin texture can be provided, for example, by decreasing pore size, reducing skin roughness, reducing the presence and/or size of wrinkles, combinations of these and the like.

The treatment period is ideally of sufficient time for the low-pH composition to improve the appearance and/or function of the target portion of skin. The treatment period typically lasts for at least 1 week (e.g., about 2 weeks, 4 weeks, 8 weeks, or even 12 weeks). In some instances, the treatment period may extend over multiple months (i.e., 3-12 months). In some instances, the composition is applied most days of the week (e.g., at least 4, 5 or 6 days a week), at least once a day or even twice a day during a treatment period of at least 2 weeks, 4 weeks, 8 weeks, or 12 weeks.

The step of applying the composition herein may be accomplished by localized application. In reference to application of the composition, the terms “localized”, “local”, or “locally” mean that the composition is delivered to the targeted area (e.g., a psoriatic plaque) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### METHODS

### Saturated Solubility Determination

This method provides a way to determine the saturated solubility of HCA compositions, using UV-Vis-Spectroscopy.

### Instruments and Materials:

A UV-VIS-NIR Spectrophotometer (such as UV-3600 Shimadzu UV-VIS-NIR Spectrophotometer) with computer data system. A suitable sample cell (such as the 1 cm path length quartz cells). A centrifuge (such as the Srvall ST40R Centrifuge), and syringe filter (such as a 0.2 um syringe filter, H-PTFE, 13 mm). 70% ethanol acidic solution (69.3% ethanol, 29.7% water, 1% HCl), pH<3.

### Saturated Solubility Sample Preparation:

The saturated solubility is measured by preparing samples with HCA added in excess (precipitation is still observed after mixing overnight). Adjust the solution pH to target using HCl/NaOH solution. Leave the solution mixing overnight to reach equilibrium. Aliquot the solution into a centrifuge tube and centrifuge, discarding any undissolved HCA. Filter the required sample amount through the 0.2 um syringe filter. Dilute solution using 70% ethanol acidic solution to desired dilution factor.

### Measurement:

Measurements are done per UV-Vis Spectrophotometer manufacturer guidelines. Turn on UV machine and warm up the lamp for 20 mins, ensure calibrations and standard dilution curves are generated. The calibration curve is achieved by plotting absorbance (y-axis) against known concentration (ppm, x-axis) for a set of calibration standards. For HCA, set the wavelength to 200-500 nm, scan speed: fast, single scan mode with auto sampling interval, and absorbance mode. Measure the background using 2× quartz cells with 70% ethanol acidic solution. Measure sample and read the absorbance at 311 nm (p-Coumaric Acid) or 325 nm (Ferulic Acid). Calculate the measured HCA concentration from the absorbance at 311 nm (p-Coumaric Acid) or 325 nm (Ferulic Acid) vs. the calibration curve and adjust to the final concentration based on the dilution factor.

### Calculations

### Calculation of Measured HCA Concentration in Water Phase

$$Concentration HCA ( \frac{mg}{ml} ) = \frac{abs peak \times dilution factor}{gradient of calibration curve \times 1000}$$

Peak for p-Coumaric Acid=311nm, Ferulic Acid=325nm

### HCA Crystallization

This method provides a way to determine HCA solubility in a composition by observing HCA crystals in situ. The method involves cycling the temperature of a test sample between freezing and thawing to imitate environmental conditions experienced by a skin care composition at an accelerated rate. This type of accelerated aging is commonly used in cosmetic product stability testing. HCA crystals can be detected using conventional means such as visual observation and microscopy.

A bulk sample of at least 10 g (e.g., 20 g-60 g) of the composition to be tested is placed in a suitable container that enables visual observation of the test sample (e.g., transparent plastic or glass jar). The test sample is subjected to 1 month of freeze/thaw temperature cycling to simulate environmental conditions that a skin care product may experience during shipping and storage.

This is sometimes referred to as accelerated aging. The temperature cycling involves a 1-week freeze cycle at −7° C., followed by a 1-week thaw cycle at 25° C., and then repeating this freeze/cycle for a total temperature cycling time of 1 month.

Upon completion of the accelerated aging process (i.e., 1 month of temperature cycling), transparent test samples are visually inspected in-situ in the transparent container to determine if HCA crystallization/precipitation occurred. For opaque and translucent samples, the entire test sample is removed from the container and transferred to a suitable transparent substrate (e.g., plastic film or glass plate) and formed into a thin film of no more than 1 mm thickness. The sample is covered with a second transparent substrate to inhibit the loss of volatile ingredients during inspection. A light source (e.g., LED lamp or the like) is used to backlight the sample to aid in visual inspection. HCA crystals will generally appear as a precipitate in the composition visible to the naked eye when observed by someone with 20/20 vision from 45 cm away. Any precipitate identified during visual observation may be further evaluated using a microscope capable of providing fluorescent birefringence observation with cross-polarized light to identify anisotropic crystals. Any anisotropic crystal that has a longest dimension of greater than 0.1 mm is identified as an HCA crystal and the total number of HCA crystals is recorded. A test sample that contains no more than 1 HCA crystal is considered to be “free of HCA crystals” and is recorded as a “pass.” A test sample that contains more than 1 HCA crystal, is recorded as a “fail.”

While not required, fourier-transform infrared spectroscopy (FTIR) can be used to confirm that the HCA crystal contains the appropriate hydroxycinnamic acid structure. FTIR spectroscopy techniques are well-known in the art. See, U.S. Pat. No. 10,912,857, US2020/0000697, and Fourier Transform Infrared Spectroscopy in Colloid and Interface Science, D. R. Scheuing, Ed., American Chemical Society, 225, 1991.

### Combinations

1. A low-pH, aqueous skin care composition, comprising:
   a. From about 0.1% to about 10% of a hydroxycinnamic acid (HCA); and
   b. From about 0.1% to about 10% of a niacinamide derivative having a free binding energy with HCA of −10 Kcal/mol or more;
   c. water,
   wherein the pH of the composition is less than 5.0.
2. The composition of the preceding feature, wherein the niacinamide derivative has a free binding energy with HCA of −7.5 kcal/mol or more.
3. The composition of any of the preceding features, wherein the niacinamide derivative has a free binding energy with HCA of −5 kcal/mol or more.
4. The composition of any of the preceding features, wherein the niacinamide derivative has a pyridine portion, in which a nitrogen is protonated in the composition.
5. The composition of any of the preceding features, wherein the niacinamide derivative is selected from the group consisting of methyl niacinamide, salts of methyl niacinamide, Nicotinamide Mononucleotide and Nicotinamide Adenine Dinucleotide, and mixtures thereof.
6. The composition of any of the preceding features, wherein the niacinamide derivative is selected from the group consisting of methyl niacinamide chloride, salts of methyl niacinamide, and mixtures thereof.
7. The composition of any of the preceding features, wherein the pH is from about 3.5 to less than 5.0.
8. The composition of any of the preceding features, wherein the HCA is coumaric acid.
9. The composition of any of the preceding features, wherein the composition is free of HCA crystals.

### EXAMPLES

The compositions in below Table 1 and 2 can be prepared using conventional methods of making skin care compositions. Such methods typically involve mixing of the ingredients in one or more steps to a relatively uniform state, with or without heating, cooling, application of vacuum, and the like. All exemplified amounts exclude minor materials such as diluents, preservatives, color solutions, feel modifying powders and elastomers, etc., that may be present in a commercial product unless otherwise specified. HCA may be added as a solid form and solubilized in-situ, dissolved as a premix, or supplied as a predispered raw material. A predispersed 15% solution of 4-HCA in PEG-4 (Lipobrite® from Vantage) is used for certain examples. For examples containing Lipobrite®, the 4-HCA and PEG-4 constituent levels are listed individually for clarity, with a superscript to denote the Lipobrite® raw material. The total Lipobrite® material added is the sum of the 4-HCA and PEG-4 constituents listed. All other materials are listed ‘as is’ from the suppliers and not broken down into the individual constituents.

TABLE 1

| | Comparative | |
|---|---|---|
| Ingredient | Example (i) | Ex. 1 |
| Glycerin | 4.5% | 4.5% |
| D-Panthenol | 0.5% | 0.5% |
| Progeline *1 | 0.25% | 0.25% |
| Trehalose | 0.10% | 0.10% |
| Sodium Benzoate | 0.05% | 0.05% |
| Sepimax Zen *2 | 1.20% | 1.20% |
| Dimethicone *3 | 4.00% | 4.00% |
| KF-6011 *4 | 0.10% | 0.10% |
| Eldew SL205 *5 | 3.00% | 3.00% |
| Pentylene | 3.00% | 3.00% |
| 10% NaOH | Adjust as needed | Adjust as needed |
| | to pH 4.5 | to pH 4.5 |
| Lipobrite (HCA-4/PEG-4) *6 | 3.33% | 3.33% |
| Niacinamide | 2.00% | — |
| 1-Methyl Nicotinamide Chloride | — | 2.00% |
| Water | q.s. to 100% | q.s. to 100% |
| 1 Month Freeze/Thaw −7° C./ | Fail | Pass |
| 25° C. | | |

*1 Progeline: available from Lucas Meyer Cosmetics.
*2 SEPIMAX ZEN available from Seppic
*3 DC 1503 available from Dow Corning
*4 KF-6011 available from Shin-Etsu
*5 Eldew SL205: available from Ajinomoto
*6 LIPOBRITE available from Vantage (15% 4-HCA, 85% PEG-4)

TABLE 2

| | Ex. 2 | Ex. 3 | Ex. 4 | Ex. 5 |
|---|---|---|---|---|
| Water Purified | QS to 100% | QS to 100% | QS to 100% | QS to 100% |
| GLYCERIN 99.0% | 4.5 | 4.5 | 4.5 | 4.5 |
| Dimethicone *3 | 4 | 4 | 4 | 4 |
| Niacinamide | — | — | — | 1 |
| Methyl Niacinamide Chloride | — | — | 5 | 1 |
| Nicotinamide Mononucleotide | 0.5 | — | — | — |
| Nicotinamide Adenine | — | 1 | — | — |
| Dinucleotide | | | | |
| L Lactic Acid 90% Solution | 1.8 | 1.8 | 1.8 | 1.8 |
| Sodium Lactate 60% solution | 1.3 | 1.3 | 1.3 | 1.3 |
| Sepimax Zen *2 | 1.2 | 1.2 | 1.2 | 1.2 |
| Hydrolyzed Yeast Protein | 1 | 1 | — | — |
| Panthenol (D-Panthenol Liquid) | 0.5 | 0.5 | 0.5 | 0.5 |
| Progeline *1 | 0.25 | 0.25 | — | — |
| DiSodium EDTA | 0.1 | 0.1 | 0.1 | 0.1 |
| PEG-11 Methyl Ether Dimethicone | 0.1 | 0.1 | 0.1 | 0.1 |
| (KF6011) *4 | | | | |
| Trehalose 100 | 0.1 | 0.1 | 0.1 | 0.1 |
| Sodium Benzoate, NF | 0.05 | 0.05 | 0.05 | 0.05 |
| Pentylene Glycol | 3 | 3 | 3 | 3 |
| Eldew SL-205 *5 | 3 | 3 | 3 | 3 |
| Lipobrite (HCA-4/PEG-4) *6 | 1 | 2 | 3.33 | 3.33 |

| | |
|---|---|
| 10% NaOH | Adjust as needed to pH of from 3.5 to less than 5 |

*1 Progeline: available from Lucas Meyer Cosmetics.
*2 SEPIMAX ZEN available from Seppic
*3 DC 1503 available from Dow Corning
*4 KF-6011 available from Shin-Etsu
*5 Eldew SL205: available from Ajinomoto
*6 LIPOBRITE available from Vantage (15% 4-HCA, 85% PEG-4)

Ex. 1 through Ex. 5 are the examples of the present disclosure. The examples of the present disclosure provide reduced crystal formation, compared to the comparative example.

The compositions shown in Table 1 were subjected to 2 freeze/thaw cycles at −7° C./25° C. for 1 week/1 week, totaling one month of temperature cycling. As can be seen in Table 1, Ex. 1 which is an example of the present disclosure containing methyl niacinamide chloride is free of HCA crystals (“pass”), while the comparative example (i) containing niacinamide demonstrate crystal formation (“fail”).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A low-pH, aqueous skin care composition, comprising:
a. from about 0.1% to about 10% of a hydroxycinnamic acid (HCA); and
b. from about 0.1% to about 10% of a niacinamide derivative having a free binding energy with HCA of −10 Kcal/mol or more;
c. water,
wherein the pH of the composition is less than 5.0.

2. The composition of claim 1, wherein the niacinamide derivative has a free binding energy with HCA of −7.5 kcal/mol or more.

3. The composition of claim 2, wherein the niacinamide derivative has a free binding energy with HCA of −5 kcal/mol or more.

4. The composition of claim 1, wherein the niacinamide derivative has a pyridine portion, in which a nitrogen is protonated in the composition.

5. The composition of claim 4, wherein the niacinamide derivative is chosen from methyl niacinamide, salts of methyl niacinamide, Nicotinamide Mononucleotide and Nicotinamide Adenine Dinucleotide, or mixtures thereof.

6. The composition of claim 5, wherein the niacinamide derivative is chosen from methyl niacinamide chloride, salts of methyl niacinamide, or mixtures thereof.

7. The composition of claim 1, wherein the pH is from about 3.5 to less than 5.0.

8. The composition of claim 1, wherein the HCA is coumaric acid.

9. The composition of claim 1, wherein the composition is free of HCA crystals.
